(19) United States
(12) Patent Application Publication
(10) Pub. No.: US 2025/0073287 A1
(43) Pub. Date: Mar. 6, 2025

(54) WEIGHT LOSS SYNBIOTIC AND PREPARATION METHOD AND USE THEREOF

(71) Applicant: Renhe Global (Shanghai) Grand Health Research Institute Co., Ltd., Shanghai (CN)

(72) Inventors: Shengquan Huang, Shanghai (CN); Haiyue Sun, Shanghai (CN); Xiaohong Xu, Shanghai (CN); Xiaolan Wang, Shanghai (CN)

(21) Appl. No.: 18/588,000

(22) Filed: Feb. 27, 2024

(30) Foreign Application Priority Data
Sep. 5, 2023 (CN) 202311135738.4

(51) Int. Cl. A61K 35/747; A61K 35/00; A61K 36/605; A61P 3/04

(52) U.S. Cl. CPC A61K 35/747; A61K 36/605; A61P 3/04; A61K 2035/115; A61K 2236/15; A61K 2236/19; A61K 2236/39; A61K 2236/51; A61K 2236/53

(57) ABSTRACT

The present application belongs to the field of synbiotic compositions, in particular discloses a weight loss synbiotic and a preparation method and use thereof. The weight loss synbiotic of the present application comprises the following components by weight: 1-20 parts of Lactobacillus paracasei HCS17-040 and 10-80 parts of mulberry leaf extract. The Lactobacillus paracasei HCS17-040 and the self-made mulberry leaf extract in the weight loss synbiotic of the present application produce a synergistic effect between them, so that the synbiotic of the present application has excellent weight loss and fat reduction effects.

### CROSS-REFERENCE TO RELATED APPLICATION

The present application claims priority from Chinese Patent Application No. 202311135738.4 filed on Sep. 5, 2023, the contents of which are incorporated herein by reference in their entirety.

### TECHNICAL FIELD

The present application belongs to the field of synbiotic compositions, in particular to a weight loss synbiotic, and a preparation method and use thereof.

### BACKGROUND

Obesity is one of the most important public health problems all over the world, which can lead to a series of serious complications, such as “trihypers”, i.e., hyperlipidemia, hyperglycemia and hypertension, and harm human health. With the development of economic level and the improvement of living standard, the obesity problem has become increasingly prominent. Studies have shown that the composition of intestinal flora of obese people, especially the proportions of firmicutes and bacteroides in the intestine, is different from that of normal weight people, and the intestinal flora is closely related to obesity. Compared with medicines and surgeries, microecological preparations have less toxic side effects and can regulate the body's energy metabolism by improving the composition of intestinal flora. Microecological preparations mainly include probiotics, prebiotics and synbiotics. Among them, probiotics can regulate intestinal flora, and prebiotic can provide nutritional substrates for probiotic. Synbiotics refer to a mixture composed of live microorganisms and substrates selectively used by microorganisms in the host, which is a substance conducive to the health of the host. Synbiotics are composed of appropriate probiotics and prebiotics, which can selectively stimulate the proliferation of specific natural bacterial strains in the gastrointestinal tract and restore the balance of intestinal flora, thereby playing an anti-obesity role. Generally speaking, prebiotics used to form synbiotic are mostly traditional prebiotics, for example, saccharides, such as inulin, fructooligosaccharides and galactooligosaccharides. However, prebiotics can only be used as the nutrient for probiotic, and have no weight loss effect itself. Therefore, finding a new prebiotic which has the effect of weight loss and can be used as the nutrient for probiotics, to prepare a weight loss synbiotic, is one of the feasible ways to further improve weight loss and fat reduction effects of the synbiotic.

### SUMMARY

In view of the problem that the traditional prebiotics involved in the prior art can only be used as the nutrient for probiotics and have no weight loss effect itself, resulting in poor weight loss effect of synbiotics, the present application provides a weight loss synbiotic and a preparation method and use thereof, so as to further improve weight loss and fat reduction effects of the synbiotic.

In order to achieve the above object, the following technical solutions are included:
A weight loss synbiotic, comprising the following components by weight: 1-20 parts of Lactobacillus paracasei HCS17-040 and 10-80 parts of a mulberry leaf extract;
a preparation method of the mulberry leaf extract comprises the following steps:
(1) crushing and sieving dried mulberry leaves, adding water and adjusting a pH value of a system to 1.5 to 4, adding an acidic protease, and stirring at 50° C. to 70° C. for extraction, and a solid-liquid separation to obtain a first filter residue and a first filtrate;
(2) adding water into the first filter residue, stirring at 50° C. to 70° C. for extraction, and performing a solid-liquid separation to obtain a second filter residue and a second filtrate;
(3) adding water into the second filter residue, adjusting a pH value of a system to 7 to 9, stirring at 50° C. to 70° C. for extraction, and performing a solid-liquid separation to obtain a third filter residue and a third filtrate; and
(4) combining the first filtrate, the second filtrate and the third filtrate, adding activated carbon for decolorization, and then successively performing centrifugation, electrodialysis desalination, reverse osmosis concentration and spray drying to obtain the mulberry leaf extract.

In a preferred embodiment of the present application, the weight loss synbiotic comprises the following components by weight: 5-10 parts of Lactobacillus paracasei HCS17-040 and 25-75 parts of the mulberry leaf extract.

The Lactobacillus paracasei HCS17-040 and the self-made mulberry leaf extract in the weight loss synbiotic of the present application produce a synergistic effect between them, so that the synbiotic of the present application has excellent weight loss and fat reduction effects.

In the preparation of mulberry leaf extract, the pH value of the system is adjusted to facilitate the dissolution of alkaloids, and protein is subjected to enzymolysis by an enzyme, so that other components are more easily dissolved, resulting in the prepared mulberry leaf extract having a higher content of active ingredients.

In a preferred embodiment of the present application, in step (1), a volume ratio of the mulberry leaves to water is 1:5-15.

In a preferred embodiment of the present application, in step (1), the extraction is conducted for 1-3 h.

In a preferred embodiment of the present application, in step (1), a sieve mesh number for the sieving is 50-150 mesh.

In a preferred embodiment of the present application, in step (1), the acidic protease has an enzyme activity of 500,000 to 1,000,000.

In a preferred embodiment of the present application, in step (2), a volume ratio of the first filter residue to water is 1:5-15.

In a preferred embodiment of the present application, in step (2), the extraction is conducted for 1-3 h.

In a preferred embodiment of the present application, in step (3), a volume ratio of the second filter residue to water is 1:5-15.

In a preferred embodiment of the present application, in step (3), the extraction is conducted for 1-3 h.

In a preferred embodiment of the present application, in step (4), the decolorization is conducted at a temperature of 35° C. to 50° C., and the decolorization is conducted for 30-120 min.

In a preferred embodiment of the present application, in step (4), an amount of the activated carbon is 0.5-3% of a total liquid mass.

In a preferred embodiment of the present application, in step (4), the centrifugation is performed at a rotational speed of 1,000-10,000 r/min.

In a preferred embodiment of the present application, in step (4), the electrodialysis desalination is performed at a voltage of 10-30 V, and the electrodialysis desalination is performed for 30-120 min.

In a preferred embodiment of the present application, in step (4), the reverse osmosis concentration is performed until Brix reaches 28%.

In a preferred embodiment of the present application, in step (4), an air inlet temperature for the spray drying is 170 to 180° C., and an air outlet temperature for the spray drying is 70 to 80° C.

A preparation method of the weight loss synbiotic comprises the following step: mixing Lactobacillus paracasei HCS17-040 and the mulberry leaf extract evenly to obtain the weight loss synbiotic.

The present application also provides use of the weight loss synbiotic in the preparation of medicines having weight loss and fat reduction effects.

Compared with the prior art, the present application has the following beneficial effects: Lactobacillus paracasei HCS17-040 and the self-made mulberry leaf extract in the weight loss synbiotic of the present application produce a synergistic effect between each other, so that the synbiotic of the present application has excellent weight loss and fat reduction effects.

### DETAILED DESCRIPTION

In order to better explain the objectives, technical solutions and advantages of the present application, the present application is further explained with specific examples below. Test methods used in examples and/or comparative examples are conventional methods unless otherwise specified. Materials, reagents, etc. used can be obtained from commercial sources unless otherwise specified.

Carbon-deficient MRS broth liquid medium: 10 g of peptone, 5 g of yeast extract, 10 g of beef extract powder, 2 g of K₂HPO₄, 5 g of CH₃COONa·3H₂O, 2 g of triammonium citrate, 0.1 g of MgSO₄·7H₂O, 0.05 g of MnSO₄·4H₂O, and 1 mL of Tween 80 are dissolved in deionized water and balanced to a total volume of 1 L, and sterilized at 121° C. for 15 min;

MRS broth liquid medium: 10 g of peptone, 5 g of yeast extract, 10 g of beef extract powder, 20 g of glucose; 2 g of K₂HPO₄, 5 g of CH₃COONa·3H₂O, 2 g of triammonium citrate, 0.1 g of MgSO₄·7H₂O, 0.05 g of MnSO₄·4H₂O, and 1 mL of Tween 80 are dissolved in deionized water and balanced to a total volume of 1 L, and sterilized at 121° C. for 15 min;

MRS solid medium is purchased from Beijing Land Bridge Technology Co., LTD.

### Example 1

### Preparation of Mulberry Leaf Extract:

(1) 1 kg of dried mulberry leaves (purchased from Shandong Huabin Biotechnology Co., LTD.) were taken, crushed and sieved through a 100-mesh sieve, 8 times amount of deionized water was added, then 30% hydrochloric acid was added to adjust a pH=2.0, 0.1 g of food-grade acidic protease FDG-22 (purchased from Ningxia Xia Sheng Industrial Group Co., LTD., with an enzyme activity of 500,000) was added, and a temperature was maintained at 55° C. to 60° C. for extraction under stirring for 1.5 h, a reaction solution was filtered to obtain an acidic extraction residue and a first filtrate, and the first filtrate was temporarily stored;
(2) 8 times amount of deionized water was added to the acidic extraction residue, a temperature was maintained at 55° C. to 60° C. for extraction under stirring for 1.5 h, a reaction solution was filtered to obtain a neutral extraction residue and a second filtrate, and the second filtrate was temporarily stored;
(3) 8 times amount of deionized water was added to the neutral extraction residue, 30% concentration of sodium hydroxide aqueous solution was added under stirring to adjust a pH=8.3, a temperature was maintained at 55° C. to 60° C. for extraction under stirring for 1.5 h, a reaction solution was filtered to obtain a filter residue and a third filtrate, and the third filtrate was temporarily stored, the filter residue was discharged;
(4) the first filtrate, the second filtrate and the third filtrate were combined, a pH was adjusted to 7.2, activated carbon with an amount of 1% of a liquid mass was added, a resulting solution was heated up to 45° C. under stirring for 45 min for decolorization, and after decolorization, a reaction solution was centrifuged by a tubular bowl centrifuge at a rotational speed of 6,000 r/min, to obtain a light brown clarified centrifuged liquid;
(5) the centrifuged liquid was subjected to electrodialysis for 40 minutes in electrodialysis equipment at a constant voltage of 20V, chloride ions in an effluent were detected, the electrodialysis was performed until there was no obvious white precipitation upon a nitric acid-silver nitrate solution was added dropwise, and an electrodialysis desalination solution was obtained;
(6) the electrodialysis desalination solution was subjected to reverse osmosis concentration in reverse osmosis membrane equipment at a low temperature (60° C. or below), and the reverse osmosis concentration was performed until a concentrated solution had Brix of 28%; and
(7) the concentrated solution was subjected spray drying, with an air inlet temperature controlled at 170° C. to 180° C. and an air outlet temperature controlled at 70° C. to 80° C., to obtain 262 g of a yellow-brown solid powder.

### Example 2

Proliferation Culture of Lactobacillus paracasei HCS17-040

2×10¹¹ CFU/g Lactobacillus paracasei HCS17-040 powder, purchased from Jiangxi Renren Health Micro-Ecological Technology Co., Ltd., was dissolved in as less MRS medium as possible, then inoculated in 10 mL MRS liquid medium, and subjected to static incubation. After static incubation at 37° C. for 24 h, plate streaking was performed, and a single colony on the plate was inoculated in MRS liquid medium and cultured at 37° C. for 24 h for microscopic examination, and the pure bacterial solution was confirmed and stored with 20% glycerin in the refrigerator at −80° C.

The mulberry leaf extract in Example 1 and a mulberry leaf extract purchased from Hunan Hill Natural Pharmaceutical Co., Ltd. were respectively dissolved in sterile water to prepare 10% (m/v) mulberry leaf extract solution and filtered by a 0.22 μm aqueous phase filter. 1 mL of a filtrate was added into 9 mL of carbon-deficient MRS liquid medium so that the final concentration of mulberry leaf extract in the medium was 1% (m/v). 1 mL sterile water, instead of a mulberry leaf extract solution, was added into 9 mL carbon-deficient MRS liquid medium as a blank control group. 1 mL sterile water was added into 9 mL MRS liquid medium as a positive control group. The activated Lactobacillus paracasei HCS17-040 strain was inoculated into the above liquid media at an inoculation amount of 2%, and cultured at 37° C. for 48 h. After 48 h, samples were taken to determine viable bacteria count in the fermentation liquid by the plate counting method, and the improvement effect of mulberry leaf extract on proliferation of Lactobacillus paracasei HCS17-040 was observed. The results are shown in Table 1.

TABLE 1
Effect of mulberry leaf extract on viable bacteria
count of Lactobacillus paracasei HCS17-040

| Group | Viable bacteria count (log CFU/mL) |
|---|---|
| Blank control group | 8.22 ± 0.05^{c} |
| Positive control group | 8.81 ± 0.06^{b} |
| Commercially purchased | 8.77 ± 0.06^{b} |
| Mulberry leaf extract | |
| Example 1 | 9.17 ± 0.04^{a} |

Note:
values were expressed as average ± SD.
Different letters in the same column indicated significant differences.

As shown in Table 1, both glucose and mulberry leaf extract can significantly promote the growth of Lactobacillus paracasei HCS17-040 (p<0.0001); there was no significant difference in the viable bacteria count of Lactobacillus paracasei HCS17-040 between the positive control group and the commercially purchased mulberry leaf extract group, and the viable bacteria count of Lactobacillus paracasei HCS17-040 of Example 1 group was significantly higher than that of the positive control group (p<0.0001), indicating that mulberry leaf extract could replace glucose as the carbon source required for the growth of Lactobacillus paracasei HCS17-040. The viable bacteria count of Lactobacillus paracasei HCS17-040 in Example 1 group was significantly higher than that of commercially purchased mulberry leaf extract group (p<0.0001), indicating that the self-made mulberry leaf extract provided by this application can significantly promote the growth of Lactobacillus paracasei HCS17-040 compared with the existing mulberry leaf extract on the market.

### Example 3

This example provided a weight loss synbiotic, which was composed of the following components, Lactobacillus paracasei HCS17-040 powder and the self-made mulberry leaf extract in Example 1; wherein in the synbiotic, Lactobacillus paracasei HCS17-040 powder accounted for 5 parts by weight, and the mulberry leaf extract accounted for 25 parts by weight.

### Example 4

This example provided a weight loss synbiotic, which was composed of the following components, Lactobacillus paracasei HCS17-040 powder and the self-made mulberry leaf extract in Example 1; wherein in the synbiotic, Lactobacillus paracasei HCS17-040 powder accounted for 7.5 parts by weight, and the mulberry leaf extract accounted for 50 parts by weight.

### Example 5

This example provided a weight loss synbiotic, which was composed of the following components, Lactobacillus paracasei HCS17-040 powder and the self-made mulberry leaf extract in Example 1; wherein in the synbiotic, Lactobacillus paracasei HCS17-040 powder accounted for 10 parts by weight, and the mulberry leaf extract accounted for 75 parts by weight.

### Comparative Example 1

A weight loss probiotic was provided, containing Lactobacillus paracasei HCS17-040 powder alone in the same amount as that of Example 5.

### Comparative Example 2

A weight loss prebiotic was provided, containing the self-made mulberry leaf extract in Example 1 alone in the same amount as that of Example 5.

### Comparative Example 3

A weight loss synbiotic was provided, wherein Lactobacillus paracasei HCS17-040 powder used in Example 5 was replaced by Lactobacillus rhamnosus HCS01-013 powder purchased from Jiangxi Renren Health Micro-Ecological Technology Co., LTD.

### Comparative Example 4

A weight loss synbiotic was provided, wherein the self-made mulberry leaf extract used in Example 5 was replaced by a mulberry leaf extract purchased from Hunan Hill Natural Pharmaceutical Co., LTD.

### Comparative Example 5

A weight loss synbiotic was provided, wherein Lactobacillus paracasei HCS17-040 powder used in Example 5 was replaced by Lactobacillus rhamnosus HCS01-013 powder purchased from Jiangxi Renren Health Micro-Ecological Technology Co., LTD, and the self-made mulberry leaf extract used in Example 5 was replaced by a mulberry leaf extract purchased from Hunan Hill Natural Pharmaceutical Co., LTD.

The functional animal experiments of synbiotic for improving obesity provided by the present application were as follows:

### 1. Establishment and Grouping of Rat Hyperlipidemia Model

According to the test method of weight loss function in “Evaluation Method of Weight Loss Function (2012) No. 107” of China Food and Drug Administration, the obese rat model was established according to the obesity prevention model, was divided into a normal control group and a model group, and given ordinary feed and high-calorie feed to make the model. After 2 weeks, the weight gain of the model group was sequenced, ⅓ obesity-resistant rats with lower weight gain were excluded, and the rest were used as further experimental animals.

The further experimental animals were randomly divided into experimental groups (for Examples 3-5 and Comparative Examples 1-5, with a total of 8 groups), a high-fat control group and a normal control group, and a number of rats for each group was guaranteed to be 10. The experimental groups and the high-fat control group were given high-fat diet, and were respectively subjected to intragastric administration of equal amounts of a test sample solution and distilled water, wherein the test sample solution was made by dissolving the test sample in 1 mL of distilled water, and the dosage was 0.125 g/kg BW day (which was equivalent to 10 times the proposed human dose that was 0.75 g/60 kg BW day).

### 2. Treatment of Rats

Body weight was measured once a week for 6 weeks. After 6 weeks, a body fat weight and a fat/body weight ratio were detected. After the detection, fasting was performed for 10 hours. For each of the rats, blood was collected from the abdominal aorta, serum was separated, and the contents of total cholesterol (TC), triglyceride (TG), high density lipoprotein cholesterol (HDL-C) and low density lipoprotein cholesterol (LDL-C) were detected by an enzyme-assay kit. 24 h after the final rat subject was administrated, feces of the rats were taken aseptically and analyzed for the microflora composition to explore the effect of the synbiotic provided by the present application on the intestinal microflora of obese rats.

### 3. Test Results

### 3.1 Results of Obesity Model Establishment

TABLE 2
Obesity model indexes of blank control group and model control group

| Group | Body weight (g) | Fat weight (g) | Fat/body weight ratio (%) |
|---|---|---|---|
| Normal control | 226.50 ± 8.46 | 9.91 ± 0.66 | 4.37 ± 0.13 |
| group | | | |
| Model control group | 255.75 ± 7.91**** | 22.58 ± 1.33**** | 8.81 ± 0.64**** |

Note:
Compared with the normal control group,
****p < 0.0001.

As can be seen from Table 2, after 2 weeks of high-fat diet, compared with the normal control group, the body weight, fat weight and fat/body weight ratio of rats in the model control group were significantly increased (p<0.0001), and the obesity model was successfully established.

### 3.2 Effects of Intervention on Body Weight, Fat Weight and Fat/Body Weight Ratio of Rats

TABLE 3
Body weight, fat weight and fat/body weight
ratio of rats before and after intervention

| | | week 6 | | |
|---|---|---|---|---|
| | | Body weight | Fat weight | Fat/body weight |
| Group | week 0 | (g) | (g) | ratio (%) |
| Normal | 251.72 ± 9.66 | 390.97 ± 13.55 | 19.16 ± 1.83 | 4.89 ± 0.30 |
| control group | | | | |
| Model | 258.53 ± 11.53 | 486.03 ± 11.47 | 70.57 ± 8.16 | 14.52 ± 1.68 |
| control group | | | | |
| Example 3 | 257.64 ± 8.50 | 425.46 ± 15.21 | 34.29 ± 2.98 | 8.06 ± 0.70 |
| Example 4 | 254.92 ± 10.74 | 406.41 ± 11.87 | 26.66 ± 3.90 | 6.54 ± 0.98 |
| Example 5 | 253.98 ± 9.95 | 391.29 ± 12.15 | 20.54 ± 3.32 | 5.25 ± 0.85 |
| Comparative | 256.47 ± 11.14 | 464.90 ± 16.79 | 52.81 ± 5.07 | 11.36 ± 1.09 |
| example 1 | | | | |
| Comparative | 252.89 ± 10.26 | 466.42 ± 14.62 | 55.71 ± 5.79 | 11.94 ± 1.24 |
| example 2 | | | | |
| Comparative | 250.95 ± 12.31 | 449.30 ± 10.23 | 45.42 ± 2.02 | 10.11 ± 0.45 |
| example 3 | | | | |
| Comparative | 259.37 ± 9.70 | 443.29 ± 12.69 | 42.87 ± 2.75 | 9.67 ± 0.62 |
| example 4 | | | | |
| Comparative | 258.17 ± 11.90 | 468.81 ± 14.65 | 56.54 ± 5.77 | 12.06 ± 1.23 |
| example 5 | | | | |

As can be seen from Table 3, compared with the model group, the body weight, fat weight and fat/body weight ratio of rats in each of the Example groups and Comparative example groups were significantly reduced (p<0.05), indicating that the synbiotic provided in each of the Example groups and Comparative example groups could significantly delay the weight gain of rats and effectively control the accumulation. Compared with the ordinary/model control group and Comparative examples 1-2 groups, the body weight, fat weight and fat/body weight ratio of rats in the Example 5 group were reduced, and the differences were significant (p<0.005), indicating that the Lactobacillus paracasei HCS17-040 and mulberry leaf extract provided by the application produced a synergistic effect between each other; and the final effects of the weight loss synbiotics of the comparative examples which contain Lactobacillus paracasei HCS17-040 and mulberry leaf extract alone, or are obtained by replacing the mulberry leaf extract prepared in Example 1 with a mulberry leaf extract purchased from Hunan Hill Natural Pharmaceutical Co., LTD and/or Lactobacillus paracasei HCS17-040 with Lactobacillus rhamnosus HCS01-013 were significantly worse than those of examples of the present application. It was also found that, the self-made mulberry leaf extract of the present application can significantly promote the proliferation of Lactobacillus paracasei HCS17-040, and the proliferation degree of Lactobacillus paracasei HCS17-040 was higher than that of Lactobacillus rhamnosus HCS01-013, indicating that Lactobacillus paracasei HCS17-040 and mulberry leaf extract produced a synergistic effect between each other. Because of the unique extraction process of the present application, the final contents of the effective substances were different from that of the existing commercially purchased mulberry leaf extract, especially the contents of polysaccharide, alkaloid and other effective substances in the self-made mulberry leaf extract were significantly different from those of commercially purchased mulberry leaf extract. As a result, the weight loss effect of the synbiotic containing self-made mulberry leaf extract was better than that of the synbiotic containing commercially purchased a mulberry leaf extract. In Examples 3-5, the body weight, fat weight and fat/body weight ratio of the rats in Examples 5 were the lowest, and the differences were significant (p<0.05), indicating that this example was the optimal example of the present application and had the best weight loss effect.

### 3.3 Effects of Intervention on Serum Biochemical Indexes of Rats

TABLE 4
Serum biochemical indexes of rats after intervention

| | TC | TG | HDL-C | LDL-C |
|---|---|---|---|---|
| Group | (mmol/L) | (mmol/L) | (mmol/L) | (mmol/L) |
| Normal control group | 1.34 ± 0.12 | 0.72 ± 0.17 | 0.96 ± 0.08 | 0.19 ± 0.09 |
| Model control group | 2.28 ± 0.18 | 1.78 ± 0.04 | 1.12 ± 0.03 | 0.31 ± 0.02 |
| Example 3 | 1.57 ± 0.14 | 0.99 ± 0.03 | 0.95 ± 0.04 | 0.24 ± 0.05 |
| Example 4 | 1.55 ± 0.16 | 0.87 ± 0.08 | 0.93 ± 0.02 | 0.21 ± 0.08 |
| Example 5 | 1.37 ± 0.13 | 0.75 ± 0.01 | 0.92 ± 0.07 | 0.20 ± 0.01 |
| Comparative example 1 | 1.78 ± 0.13 | 1.34 ± 0.12 | 1.01 ± 0.05 | 0.28 ± 0.03 |
| Comparative example 2 | 1.85 ± 0.11 | 1.15 ± 0.10 | 1.06 ± 0.09 | 0.27 ± 0.07 |
| Comparative example 3 | 1.82 ± 0.15 | 1.18 ± 0.13 | 0.99 ± 0.06 | 0.22 ± 0.04 |
| Comparative example 4 | 1.78 ± 0.12 | 1.28 ± 0.07 | 1.02 ± 0.01 | 0.25 ± 0.06 |
| Comparative example 5 | 1.87 ± 0.10 | 1.31 ± 0.08 | 1.00 ± 0.10 | 0.26 ± 0.01 |

It can be seen from Table 4 that the synbiotic provided by the present application can effectively reduce the contents of TC and TG in rats, so as to effectively prevent hyperlipidemia and other related diseases. Among them, Example 5 has the lowest TC, TG, LDL-C and HDL-C values (p<0.0001), indicating that the fat reduction effect was extremely significant, and this example was the optimal example of the present application.

3.4 Bacteria Count in Rats after Intervention

TABLE 5
Bacteria count in rats after intervention (log CFU/g)

| | Total bacteria | | | | Faecalibacterium |
|---|---|---|---|---|---|
| Group | count | Bacteroides | bifidobacterium | lactobacillus | prausnitzii |
| Normal control group | 11.61 ± 0.10 | 10.34 ± 0.07 | 6.30 ± 0.01 | 7.25 ± 0.09 | 9.10 ± 0.08 |
| Model control group | 10.38 ± 0.16 | 9.71 ± 0.10 | 6.11 ± 0.02 | 6.74 ± 0.02 | 8.86 ± 0.06 |
| Example 3 | 10.79 ± 0.06 | 10.21 ± 0.08 | 7.33 ± 0.10 | 7.64 ± 0.04 | 9.97 ± 0.05 |
| Example 4 | 10.89 ± 0.03 | 10.33 ± 0.05 | 7.49 ± 0.03 | 7.86 ± 0.07 | 10.10 ± 0.07 |
| Example 5 | 11.18 ± 0.07 | 10.44 ± 0.02 | 7.60 ± 0.06 | 7.98 ± 0.06 | 10.22 ± 0.03 |
| Comparative | 10.55 ± 0.06 | 9.86 ± 0.04 | 7.24 ± 0.04 | 7.26 ± 0.01 | 9.70 ± 0.09 |
| example 1 | | | | | |
| Comparative | 10.66 ± 0.02 | 9.81 ± 0.09 | 7.18 ± 0.09 | 7.21 ± 0.03 | 9.85 ± 0.01 |
| example 2 | | | | | |
| Comparative | 10.52 ± 0.08 | 10.06 ± 0.01 | 7.14 ± 0.05 | 7.44 ± 0.05 | 9.80 ± 0.09 |
| example 3 | | | | | |
| Comparative | 10.51 ± 0.05 | 10.11 ± 0.04 | 7.18 ± 0.01 | 7.46 ± 0.08 | 9.86 ± 0.02 |
| example 4 | | | | | |
| Comparative | 10.53 ± 0.09 | 9.79 ± 0.08 | 7.16 ± 0.04 | 7.16 ± 0.02 | 9.72 ± 0.03 |
| example 5 | | | | | |

As can be seen from Table 5, compared with the normal control group, the total bacterial count, and the amounts of bacteroides, bifidobacterium, lactobacillus and Faecalibacterium prausnitzii in the model control group all decreased to varying degrees, indicating that obesity has a significant impact on the intestinal flora of rats; compared with the model control group, the total bacterial count, and the numbers of bacteroides, bifidobacterium, lactobacillus and Faecalibacterium prausnitzii in Examples 3-5 and Comparative examples 1-5 increased to varying degrees. The top three increases in total bacterial count and different bacterial genera were Examples 5, 4 and 3, and the numbers of bifidobacterium, lactobacillus and Faecalibacterium prausnitzii in some examples were even higher than those in the normal control group, indicating that Examples 3-5 and Comparative examples 1-5 groups, especially Examples 3-5 groups, can significantly improve the intestinal flora disturbance caused by obesity in rats. In summary, Example 5 group had the best effect in regulating intestinal flora, with the highest total bacterial count, and the highest numbers of bacteroides, bifidobacterium, lactobacillus and Faecalibacterium prausnitzii.

Finally, it should be noted that the above examples are used only to illustrate the technical solutions of the present application and not to limit the scope of protection of the present application. Although the present application is described in detail with reference to the preferred example, the ordinary person skilled in the art should understand that the technical solutions of the present application can be modified or equivalently replaced without deviating from the substance and scope of the technical solution of the present application.

1. A weight loss synbiotic, comprising the following components by weight: 1-20 parts of Lactobacillus paracasei HCS17-040 and 10-80 parts of a mulberry leaf extract;
a preparation method of the mulberry leaf extract comprises the following steps:
(1) crushing and sieving dried mulberry leaves, adding water and adjusting a pH value of a system to 1.5 to 4, adding an acidic protease and stirring at 50° C. to 70° C. for extraction, and performing a solid-liquid separation to obtain a first filter residue and a first filtrate;
(2) adding water into the first filter residue, stirring at 50° C. to 70° C. for extraction, and performing a solid-liquid separation to obtain a second filter residue and a second filtrate;
(3) adding water into the second filter residue, adjusting a pH value of a system to 7 to 9, stirring at 50° C. to 70° C. for extraction, and performing a solid-liquid separation to obtain a third filter residue and a third filtrate; and
(4) combining the first filtrate, the second filtrate and the third filtrate, adding an activated carbon for decolorization, and then successively performing centrifugation, electrodialysis desalination, reverse osmosis concentration and spray drying to obtain the mulberry leaf extract.

2. The weight loss synbiotic according to claim 1, wherein the weight loss synbiotic comprises the following components by weight: 5-10 parts of Lactobacillus paracasei HCS17-040 and 25-75 parts of the mulberry leaf extract.

3. The weight loss synbiotic according to claim 1, wherein at least one of the following is involved:
in step (1), a volume ratio of the mulberry leaves to water is 1:5-15;
in step (2), a volume ratio of the first filter residue to water is 1:5-15; and
in step (3), a volume ratio of the second filter residue to water is 1:5-15.

4. The weight loss synbiotic according to claim 1, wherein at least one of the following is involved:
in step (1), the extraction is conducted for 1-3 h;
in step (2), the extraction is conducted for 1-3 h; and
in step (3), the extraction is conducted for 1-3 h.

5. The weight loss synbiotic according to claim 1, wherein in step (1), a sieve mesh number for the sieving is 50-150 mesh.

6. The weight loss synbiotic according to claim 1, wherein in step (1), the acidic protease has an enzyme activity of 500,000 to 1,000,000.

7. The weight loss synbiotic according to claim 1, wherein in step (4), the decolorization is conducted at a temperature of 35° C. to 50° C., and the decolorization is conducted for 30-120 min.

8. The weight loss synbiotic according to claim 1, wherein at least one of the following is involved:
in step (4), an amount of the activated carbon is 0.5-3% of a total liquid mass;
in step (4), the centrifugation is performed at a rotational speed of 1,000-10,000 r/min;
in step (4), the electrodialysis desalination is performed at a voltage of 10-30 V, and the electrodialysis desalination is performed for 30-120 min;
in step (4), the reverse osmosis concentration is performed until Brix reaches 28%; and
in step (4), an air inlet temperature for the spray drying is 170° C. to 180° C., and an air outlet temperature for the spray drying is 70° C. to 80° C.

9. A method for preparing the weight loss synbiotic according to claim 1, comprising the following step: mixing Lactobacillus paracasei HCS17-040 and the mulberry leaf extract evenly to obtain the weight loss synbiotic.

10. A method for preparing the weight loss synbiotic according to claim 2, comprising the following step: mixing Lactobacillus paracasei HCS17-040 and the mulberry leaf extract evenly to obtain the weight loss synbiotic.

11. A method for preparing the weight loss synbiotic according to claim 3, comprising the following step: mixing Lactobacillus paracasei HCS17-040 and the mulberry leaf extract evenly to obtain the weight loss synbiotic.

12. A method for preparing the weight loss synbiotic according to claim 4, comprising the following step: mixing Lactobacillus paracasei HCS17-040 and the mulberry leaf extract evenly to obtain the weight loss synbiotic.

13. A method for preparing the weight loss synbiotic according to claim 5, comprising the following step: mixing Lactobacillus paracasei HCS17-040 and the mulberry leaf extract evenly to obtain the weight loss synbiotic.

14. A method for preparing the weight loss synbiotic according to claim 6, comprising the following step: mixing Lactobacillus paracasei HCS17-040 and the mulberry leaf extract evenly to obtain the weight loss synbiotic.

15. A method for preparing the weight loss synbiotic according to claim 7, comprising the following step: mixing Lactobacillus paracasei HCS17-040 and the mulberry leaf extract evenly to obtain the weight loss synbiotic.

16. A method for preparing the weight loss synbiotic according to claim 8, comprising the following step: mixing Lactobacillus paracasei HCS17-040 and the mulberry leaf extract evenly to obtain the weight loss synbiotic.

17. Use of the weight loss synbiotic according to claim 1 in preparation of medicines having weight loss and fat reduction effects.
